(19) United States
(12) Patent Application Publication
(10) Pub. No.: US 2012/0288480 A1
(43) Pub. Date: Nov. 15, 2012

(54) CELL POPULATION COMPRISING ORBITAL FAT-DERIVED STEM CELLS (OFSCS) AND THEIR ISOLATION AND APPLICATIONS

(75) Inventor: Jennifer Hui-Chun Ho, Taipei City (TW)

(73) Assignee: TAIPEI MEDICAL UNIVERSITY, Taipei City (TW)

(21) Appl. No.: 13/032,056

(22) Filed: Feb. 22, 2011

(51) Int. Cl. A61K 35/12; C12N 5/071; A61P 27/02; C12N 5/0775

(52) U.S. Cl. 424/93.7; 435/366; 435/371

(57) ABSTRACT

The invention relates to a cell population comprising minimal volume of orbital fat-derived stem cells (OFSCs) and its isolation, purification, characterization and application. The OFSCs of the invention are capable of multilineage development and express at least CD90 and CD 105 but not hematopoietic and epithelial markers. The OFSCs have colony formation ability and multi-lineage differentiation ability. They possess at least osteogenic, chondrogenic and adipogenic differentiation capacity; besides mesodermal tri-linage differentiation, the OFSCs have corneal epithelial differentiation potential. Taking together, orbital fat tissues are a novel source for multi-potent stem cells which possess multiple therapeutic potential. Therefore, the OFSCs can be used in cell therapy and tissue engineering.

### FIELD OF THE INVENTION

The invention relates to a cell population comprising orbital fat-derived stem cells (OFSCs) and its isolation, purification, characterization and application. In particular, the OFSCs are capable of multilineage development and express at least CD90 and CD 105 but not hematopoietic and epithelial markers.

### BACKGROUND OF THE INVENTION

Irreversible loss of corneal epithelial cells, which result from a variety of corneal diseases, may cause corneal opacity and lead to blindness in advanced cases. Stem cell transplantation has brought along great hope for repair and regeneration of ocular tissues. So far it has been reported that stem and progenitor cells can be isolated from human eye tissues such as corneal limbal epithelium, ciliary epithelium and Müller glia. Among these achievements, the major breakthrough is autologous limbal stem cell transplantation, which can replenish the loss of corneal epithelial cells which cannot spontaneously regenerate due to limbal cell insufficiency and has been successfully used for patient treatment. However, injury to the contralateral donor site and the limited source are the major drawbacks. Besides, for patients with severe, bilateral eye diseases, limbal cell transplantation is not possible and allogenic corneoscleral graft transplantation is the only solution (Pellegrini G, De Luca M, Arsenijevic Y., Semin Cell Dev Biol. 2007; 18:805-818), yet the long-term success of allogenic corneal transplantation transplantation is still hampered by rejection in spite of routine administration of long-term immunosuppressant (Liang L, Sheha H, Tseng S C., Arch Ophthalmol. 2009; 127:1428-1434; Limb G A, Daniels J T, Cambrey A D, et al., Curr Eye Res. 2006; 31:381-390). Therefore, it is imperative to look for alternative autologous stem cell sources for corneal surface transplantation to avoid rejection and damage to the normal ocular structures.

During embryonic development, most of ocular and orbital components are derived from neuroectoderm. Neural crest cells, a transient population arise from neuroectoderm, contribute the most mesenchymal cells of the facial primordia. Neural crest cells from the diencephalon migrate to and settle around the optic vesicles during early ocular development, which make a major contribution to connective tissue components of eyes and orbit except fibers of extracellular muscles and endothelial lining of blood vessels. It is know in the art that human neural crest stem cells directly differentiated into peripheral nerve system and mesenchymal lineages. Besides, linage-tracing studies in vivo demonstrated the developmental origin for mesenchymal stem cells (MSCs) and adipocytes in neural crest.

Adipose tissue is an especially rich source of stem cells. It has been demonstrated that adipose tissue contains a population of multipotent stem cells and others have shown that this tissue is a source of endothelial cells (see U.S. Pat. No. 5,372,945). Korn et al, reported that adipose-derived stem cells were isolated from human orbital adipose tissue and they have the potential to differentiate into the adipocyte, smooth muscle, and neuronal/glial lineages (Ophthal Plast Reconstr Surg. 2009 January-February; 25(1):27-32). However, the adipose-derived stem cells reported by Korn et al express CD 34, which indicates that these cells may be hematopoietic origin.

Given the potential of stem cells derived from adipose tissue for therapeutic purposes, there is thus a need to develop novel stem cells from other adipose tissue sources.

### SUMMARY OF THE INVENTION

The invention provides a cell population which comprises orbital fat-derived stem cell (OFSCs) expressing at least CD90 and CD 105, wherein said OFSCs are not of hematopoietic and epithelial origins, and wherein said OFSCs are capable of multilineage development.

The invention also provides a method for isolation and purification of cell population comprising OFSCs of Claim 1, comprising the steps of:
(a) collecting a sample containing 0.5-2 ml of orbital fat tissues;
(b) fragmenting the orbital fat tissues and suspending the resulting tissues in a buffer solution containing an extracellular matrix (ECM)-degrading enzyme;
(c) filtering the resulting solution to obtain the pellet;
(d) re-suspending the pellet to obtain a cell suspension solution;
(e) counting the cells in the cell suspension solution and culturing the cells in medium with low seeding density of less than 8,000 cells/cm²;
(f) collecting cells with colony-formation ability and sub-culturing these cells in an non-contact manner; and
(g) identifying and charactering the resulting cells with cell surface markers and multiple differentiation ability, wherein OFSCs are the resulting cells having multilineage development and expressing at least CD90 and CD 105 but lacking hematopoietic and epithelial cell surface markers.

The invention further provides a method for differentiation of orbital fat-derived stem cells (OFSCs) to corneal epithelial cells, comprising the step of mix-culturing OFSCs with corneal epithelial cells.

The invention also further provides a method for preparing corneal epithelial cell preparations, comprising: (a) isolating orbital fat-derived stem cells (OFSCs) from orbital adipose samples; (b) mix-culturing the OFSCs with labeled corneal epithelial cells to differentiating into corneal epithelial cells; and (c) removing the labeled corneal epithelial cells to obtain the OFSCs-derived corneal epithelial cell preparations.

### BRIEF DESCRIPTION OF THE DRAWING

FIG. 1 shows morphology, growth kinetics and immunophenotypic characterization of orbital fat-derived stem cells (OFSCs). (A) OFSCs were adherent, spindle-shaped, fibroblast-like cells. (B) Under the same culture condition, the growth kinetics curve of OFSCs was comparable to bone marrow-derived mesenchymal stem cells (BM-MSCs). (n=3) (C) Surface immuno-phenotyping showed that OFSCs were mesenchymal rather than hematopoeitic or epithelial in origin. (n=3).

FIG. 2 shows in vitro osteogenic differentiation of OFSCs. (A) Under osteogenic induction for 1 week, cells expressed osteogenic marker genes including alkaline phosphatase (ALP), type I collagen α1 and α2 (Col IA1 and Col IA2), osteopontin (OP), osteonectin (ON) and osteocalcin (OC). Expression of periostin (POSTN) was not significantly different. (t-test, * P<0.05, n=3). (B) OFSC-differentiated cells with strong ALP activity were more flattened and broadened in shape at the end of first week induction. (C) OFSC-differentiated cells produced mineralized matrix, which stained positive by von Kossa stain after 3 weeks of osteogenic induction. (Cells in each picture derived from different donor).

FIG. 3 shows in vitro chondrogenic differentiation of OFSCs. (A) Up-regulation of chondrogenic marker genes such as aggrecan (ACAN), Type II α1 collagen (Col IIA1), cartilage acidic protein 1 (CRTAC1), syndecan 2 (SDC2), cartilage oligomeric matrix protein (COMP) and cartilage matrix protein matrilin (MATN1) in OFSC-differentiated cells were detected after one-week of chondrogenic induction. Expression of heparan sulfate proteoglycan 2 (HSPG2) was not significantly different. (t-test, * P<0.05, n=3) (B) Under pellet culture for six weeks, the pellets increased in size, and (C) the histological section of pellets showed the production of cartilagenous extracellular matrix under safranin O stain (Cells in each picture derived from different donors).

FIG. 4 shows in vitro adipogenic differentiation of OFSCs. (A) Under adipogenic induction, OFSC-differentiated cells expressed extremely high level of adipogenic marker genes such as peroxisome proliferator-activated receptor gamma (PPARgamma), fatty acid binding protein (aP2), fatty acid synthase (FASN), complement factor D (Adipsin) and adiponectin during the first-week of adipogenic differentiation. (t-test, * P<0.05, n=3) (B) Massive intracellular lipid droplets were evident by oil red O staining after two weeks of induction (Cells in each picture derived from different donor).

FIG. 5 shows ability of epithelial differentiation in OFSCs upon mix-culture with human corneal epithelial (HCE-T) cells but not in ADSCs. (A) OFSCs and HCE-T cells were mix-cultured in HCE-T medium. (B) ADSCs and HCE-T cells were mix-cultured in HCE-T medium. (C) Confluence of cells was noted after mix-culture for 5 days. Cobblestone-like cell islets surrounded by fibroblast-like cells were found. (D) Similar morphological changes were also observed in mix-cultured ADSCs and HCE-T cells. (E) The frequency of CD105 positive cells was not significantly different between OFSCs and ADSCs after mix-culture with HCE-T cells. (F) The frequency of ESA positive cells was significantly higher in OFSCs than in ADSCs after mix-cultured with HCE-T cells. (t-test, * P<0.05, n=3)

FIG. 6 shows epithelial differentiation of OFSCs in the mix-culture system. (A) Quantum dots from 1 to 10 nM demonstrated dose-dependent label efficiency in OFSCs. (B) In the mix-culture system, quantum dot-labeled OFSCs with red fluorescence signals could easily be distinguished from cobblestone-like HCE-T cells. (C) Quantum dot-labeled cells at the margin of cobblestone-like cell islets became oval to round in shape. (D) After 5 days of mix-culture, 20.1±0.77% of quantum dot-labeled cells also expressed ESA. (t-test, * P<0.05, n=3) (E) Zonal occluding-1 (ZO-1) was expressed on the surface of HCE-T cells. (F) OFSCs alone cultured in HCE-T medium did not express ZO-1. (G) Under mix-culture for 5 days, quantum dot-labeled cells at the margin of cobblestone-like cell islets contacted with neighborhood cells and expressed ZO-1 at intercellular junctions.

FIG. 7 shows corneal epithelial differentiation of OFSCs when mix-cultured with HCE-T cells. (A-D) Quantum dots-labeled OFSCs were mix-cultured with HCE-T cells for 5 days. Quantum dots-labeled cells at the margin of cobblestone-like cell islets (A, arrow) began to express CK19 (B, arrow). Most HCE-T cells (D) and some of quantum dots-labeled cells expressed CK3 (C and D, arrow). When OFSCs alone were cultured in HCE-T medium for 5 days, neither the morphology of those cells was altered (E, G), nor CK19 (F) and CK3 (H) expression was found.

FIG. 8 shows seldom ADSCs were able to differentiate into corneal epithelial cells. (A-D) Quantum dots-labeled ADSCs were mix-cultured with HCE-T cells for 5 days. HCE-T and ADSCs did not express CK19 (A, B), and very few quantum dot labeled cells (C, D arrow) expressed CK3 after mix-culture.

FIG. 9 shows direct contact with HCE-T cells indispensable for epithelial differentiation of OFSCs. OFSCs and HCE-T cells were co-cultured in transwell, non-contact system for 7 days. The population of CD105- and ESA-positive cells on OFSCs was not altered (A) (n=3). Negative staining of ZO-1 (B), CK19 (C) and CK3 (D) for OFSCs after transwell co-culture for 5 days was found.

### DETAILED DESCRIPTION OF THE INVENTION

Adipose tissue-derived stromal cells and cells from the stromal vascular fraction of the aspirated subcutaneous fat have been demonstrated to possess stem cell properties. The invention demonstrates the existence of multi-potent stem cells in human orbital fat tissues (OFSCs) and the multi-potent OFSCs can be obtained from a minimal amount of orbital fat tissues. The invention unexpectedly found that orbital fat is a good source to isolate stem cells having multi-potentiality, including the potential to differentiate into osteogenic, chondrogenic, adipogenic and corneal epithelial cells.

### DEFINITIONS

As used herein, “adipose tissue” refers to a tissue containing multiple cell types including adipocytes and microvascular cells. Accordingly, adipose tissue refers to fat including the connective tissue that stores the fat.

As used herein, “stem cells” are cells that possess self-renewal ability and multiple differentiation ability when exposed to specific environmental conditions. Self-renewal means that during cell division, at least one of the two daughter cells will be a stem cell.

As used herein, “multi-potent” means a cell that has the potential of differentiating into at least two cell.

As used herein, “corneal epithelia” or “corneal epithelium” is made up of epithelial tissue and covers the front of the cornea. It consists of several layers of cells. The cells of the deepest layer are columnar; then follow two or three layers of polyhedral cells, the majority of which are prickle cells similar to those found in the stratum mucosum of the cuticle.

As used herein, “differentiation” means the formation of cells expressing functional markers known to be associated with cells that are more specialized and closer to becoming terminally differentiated cells incapable of further division or differentiation.

As used herein, “lineage committed cell” means a progenitor cell that is no longer multi-potent and fated to differentiate into a specific cell lineage.

As used herein, “autologous transplant” means that the transplanted material is derived from and transplanted to the same individual.

As used herein, “proliferation” or “expansion” means an increase in cell number.

As used herein, “cell surface marker” means a protein expressed on the surface of a cell which is detectable via specific antibodies.

As used herein, “positive for expression” means that the marker of interest, whether intracellular or extracellular, is detectable in or on a cell using any method, including, but not limited to, flow cytometry. The terms “positive for expression,” “positively expressing,” “expressing,” and “+” used in superscript are used interchangeably herein.

As used herein, “negative for expression” means that the marker of interest, whether intracellular or extracellular, is not detectable in or on a cell using any method, including but not limited to flow cytometry. The terms “negative for expression,” “negative expressing”, “not expressing,” and “−” used in superscript are used interchangeably herein.

As used herein, “isolated,” used in reference to a single cell or cell population, means that the cell or cell population is substantially free of other cell types or cellular material with which it naturally occurs in the orbital fat.

### Cell Population of Orbital Fat-Derived Stem Cells (OFSCs) and a Composition Containing the Same

In one aspect, the invention provides a cell population, which comprises orbital fat-derived stem cell (OFSCs) expressing at least CD90 and CD 105, wherein said OFSCs are not of hematopoietic and epithelial origins, and wherein said OFSCs are capable of multilineage development.

According to the invention, the OFSCs are derived from orbital fat. They are useful for particular embodiments of the invention such as for reconstituting, regenerating, or repairing a disease of interest or for manufacturing kits. Orbital fat is a semifluid adipose cushion that lines the bony orbit supporting the eye. According to the invention, the OFSCs may be isolated from the stromal vascular fraction of orbital adipose tissue.

Using a combination of cell surface markers and others markers such as intracellular enzymes and the light scattering properties of the cells, stem cell grafts can be advantageously “tailored” for particular therapeutic uses. For example, stem cells that give rise to hematopoietic lineages can be used to replace hematopoietic system in bone marrow; stem cells that give rise to mesenchymal lineages can be used to repair musculoskeletal diseases; stem cells that differentiate into epithelial lineages can be used to repair surface injury including cornea; stem cells that differentiate into neuronal lineages can be used to treat neurodegenerative disease including retinal degeneration. Thus, the novel combination of cell markers disclosed herein confers advantages as identification of the stem cell sources that are functionally and quantitatively best for use in isolating stem cells.

Antibodies can be used to recognize surface molecules differentially expressed on target cells. The cell surface marker means a protein expressed on the surface of a cell, which is detectable via specific antibodies. Cell markers as well as surface markers that are useful in the invention include, but are not limited to, the CD (clusters of differentiation) antigens CD29, CD44, CD49b, CD49e, CD58, CD80, CD86, CD90, CD105, HLA-ABC, CK-19, CK-3, CD14, CD31, CD34, CD40, CD45, CD106, CD117, CD133, CD146 and HLA-DR. CD29 is an integrin 1 subunit expressed on most cells; CD49b is an integrin 2 subunit of VLA-2 receptor; CD49e is an integrin 5 subunit of fibronectin receptor; CD44 is a cell-surface glycoprotein involved in cell-cell interactions; CD58 is a cell adhesion molecule expressed on Antigen Presenting Cells (APC), particularly macrophages; CD90 is a GPI-cell anchored molecule found on prothymocyte cells; CD105 is a disulfide-linked homodimer found on endothelial cells but absent from most T and B cells; HLA-ABC are MHC class I antigens associated with 132-microglobulin and are expressed by all human nucleated cells; CK-19 is a type I keratin; CK-3 is keratin 3; CD14 is a component of the innate immune system; CD31 is a homotypic adhesion molecule found on all endothelial cells and some platelets and leukocytes; CD34 is a highly glycosylated type I transmembrane protein expressed on 1-4% of bone marrow cells; CD40 is a costimulatory protein found on antigen presenting cells; CD45 is a leukocyte common antigen found on all cells of hematopoietic origin; CD80 is a protein found on activated B cells and monocytes that provides a costimulatory signal necessary for T cell activation and survival; CD86 is a protein expressed on antigen-presenting cells that provides costimulatory signals necessary for T cell activation and survival; CD 106 is the protein encoded by the VCAM1 gene and functions as a cell adhesion molecule; CD117 is the c-kit ligand receptor found on 1-4% of bone marrow stem cells; CD133 is a pentaspan transmembrane glycoprotein expressed on primitive hematopoietic progenitor cells; and HLA-DR is the MHC Class II molecule.

In one embodiment, the OFSCs of the invention can express at least CD 90 and CD 105. In addition to CD90 and CD105, the OFSCs of the invention can express CD29, CD44, CD49b, CD49e, CD58, HLA-ABC or a combination thereof. In another embodiment, the OFSCs of the invention can express CD29, CD44, CD49b, CD49e, CD58, CD90, CD105 and HLA-ABC. The expression of CD90 and CD105 shows that the OFSCs of the invention express mesenchymal stem cell markers. The expression of CD29, CD44, CD49b, CD49e, or a combination thereof further confirms that the OFSCs are mesenchymal origins.

In some embodiments, the lack of expression of a cell surface marker defines the OFSCs of the invention. According to the invention, the OFSCs are negative for at least hematopoietic stem cell marker CD34. In addition to CD34, the OFSCs are negative for hematopoietic stem cell marker CD133, endothelial progenitor cell marker CD31, vascular cell adhesion molecule-1, CD106, vascular endothelial tight junction marker CD146, leukocyte common antigen CD45, monocyte marker CD14 and CD117 (c-kit) or a combination thereof, which indicates that these cells are not of hematopoietic origin. According to the invention, the OFSCs are negative for CD40, CD80, CD86, HLA-DR or a combination thereof, which indicates that these cells do not cause rejection reaction in a mammal.

Based on the above-mentioned unique cell surface marker signatures, individual stem cell populations having unique functional characteristics have been identified.

In some embodiments at least 70%, 80% or 90% of the OFSCs within a cell population of the invention express the cell markers of interest; in other embodiments at least 80%, or 90% of the OFSCs within the stem cell population express the cell markers of interest; in yet other embodiments at least 95%, 96%, 97%, 98%, 99%, or even 100% of the of the OFSCs within the stem cell population express the cell markers of interest. “Substantially free” means less than about 5%, 4%, 3%, 2%, 1%, or even 0% of the cells in the population expressing the marker of interest. While the isolation of purified cell population from orbital fat is specifically exemplified herein, the isolation of such cells from other sources is also contemplated.

Selective methods known in the art and described herein can be used to characterize OFSCs. Commonly, sources of OFSCs are reacted with monoclonal antibodies, and subpopulations of cells expressing cell surface antigens are either positively or negatively selected with quantum dots, immunomagnetic beads by complement mediated lysis, agglutination methods, or fluorescence activated cell sorting (FACS). The functional attributes of the resulting subpopulations with a defined cell surface phenotype are then determined using a colony-forming assay.

According to the invention, the OFSCs have colony formation ability and multi-lineage differentiation ability. Accordingly, the OFSCs of the invention possess osteogenic, chondrogenic and adipogenic differentiation capacity; besides mesodermal tri-linage differentiation, the OFSCs have corneal epithelial differentiation potential. Taken together, orbital fat tissues are a novel source for multi-potent stem cells which possess differential potential. Therefore, the OFSCs can be used in cell therapy and tissue engineering, such as tissue regeneration for degenerative disease, repair of tissue injury, organ regeneration and medical cosmetology.

The invention also provides a composition comprising the cell population of the invention. According to the invention, in addition to the cell population, the above composition may contain one or more inactivated carriers that are permitted pharmaceutically. Examples of the inactivated carriers include preservative, solublizer, stabilizer, etc. The composition may be used for non-oral administration, for example intravenous, subcutaneous, intra-peritoneal administration or topical application. A dosage of the cell population may vary in accordance with kind of disease, degree of seriousness of disease, administration route, or weight, age and sex of patient.

### Method for Isolation and Purification of OFSCs

The OFSCs of the invention can be isolated and purified from orbital fatty tissues using a variety of methods, including those described herein and exemplified below. For identification and characterization, the isolated OFSCs are positively selected by sorting for expression of cell surface markers and negatively sorting for lack of expression of cell surface markers.

In another aspect, the invention develops a facile method to isolate OFSCs from a minimal volume (around 0.5-2 ml) of orbital fatty tissues. Accordingly, the invention provides a method for isolation and purification of cell population comprising OFSCs, comprising the steps of:
(a) collecting a sample containing 0.5-2 ml of orbital fat tissues;
(b) fragmenting the orbital fat tissues and suspending the resulting tissues in a buffer solution containing an extracellular matrix (ECM)-degrading enzyme;
(c) filtering the resulting solution to obtain the pellet;
(d) re-suspending the pellet to obtain a cell suspension solution;
(e) counting the cells in the cell suspension solution and culturing the cells in medium with low seeding density of less than 8,000 cells/cm²;
(f) collecting cells with colony-formation ability and sub-culturing these cells in an non-contact manner; and
(g) identifying and charactering the resulting cells with cell surface markers and multiple differentiation ability; wherein OFSCs are the resulting cells having multilineage development and expressing at least CD90 and CD 105 but lacking hematopoietic and epithelial cell surface markers.

According to the invention, the sample containing about 0.5 to about 2.0 ml of orbital fat tissues in step (a) can be collected by directly removing the orbital tissue from intraorbital cavity or collected during blepharoplasty surgeries for entropion, ectropion, ptosis or baggy lid. The collection can be in a coagulation-free, non-aspirated manner. Preferably, the sample contains about 0.5 to about 1.5 ml or about 0.5 to about 1.0 ml of orbital fat tissues. More preferably, the sample contains about 1.0 ml of orbital fat tissues.

According to the invention, any method known in the art can be used in the fragmentation of the orbital fat tissues in step (b). For example, the orbital tissues in step (b) can be simply fragmented with scissors or forceps. After fragmentation, the resulting tissues are placed in a buffer solution containing an extracellular matrix (ECM)-degrading enzyme. Preferably, the ECM-degrading enzyme is collagenase, matrix metalloproteinase, endopeptidases or hyaluronidase. More preferably, the ECM-degrading enzyme is collagenase. More preferably, the collagenase is collagenase type I.

According to the invention, the filtration in step (c) can be performed with any method known in the art to obtain cell pellets. For example, filter, filtration membrane or strainer can be used. After filtration, centrifugation can be further performed.

According to the invention, in step (d), the cell pellet is re-suspended to obtain a cell suspension solution. Subsequently, in step (e), the cells in the cell suspension solution are counted and cultured in medium with low seeding density of less than 8,000 cells/cm². Preferably, the seeding density is from 500-8,000 cells/cm², 1,000-8,000 cells/cm² or 3,000 or 5,000 cells/cm².

According to the invention, in step (f), most initial seeded cells are dead and detached after 2-4 weeks, and the remainder (around 0.05 cells/cm²) possess colony formation ability. Cells derived from single colony are collected and maintained in suitable medium (such as Mesen Pro Medium) to increase cell numbers. Once adherent cells reach approximately 60% to 70% confluence, cells are detached and re-plated at a ratio of 1:3 under the same culture conditions.

According to the invention, in step (g), the resulting cells of step (e) are identified and characterized using the cell surface markers described herein. As a result, the cells have multilineage development at least osteogenic, adipogenic and chondrogenic differentiation ability, and express at least CD90 and CD 105 but lacks hematopoietic and epithelial cell surface markers. The expression and lack of expression of the cell markers are those as mentioned in the above section of “Cell Population of Orbital Fat-Derived Stem Cells (OFSCs).”

The invention only needs a small amount of orbital fat sample to obtain a number of multi-potent OFSCs, which provides an advantageous way for getting stem cells.

### Methods for Differentiation of OFSCs to Corneal Epithelial Cells

In a further aspect, the invention provides a method for differentiation of orbital fat-derived stem cells (OFSCs) to corneal epithelial cells, comprising the step of mix-culturing OFSCs with corneal epithelial cells.

The invention found that direct contact with corneal epithelial cells is essential for OFSCs to commit to corneal epithelial cells, suggesting their potential for regeneration of lost corneal epithelial cells on the ocular surface via contact with corneal epithelial cells.

According to the invention, the mix-culture of OFSCs with corneal epithelial cells can be performed in any appropriate medium. Preferably, media suitable for corneal epithelial cells can be used in the mix-culture of the invention. For example, Dulbecco's modified Eagle's medium (DMEM) can be used.

According to the invention, loss of CD105 expression and increased expression of epithelial cell markers (such as epithelial specific antigen and zonal occludin-1) are found upon mix-culture with corneal epithelial cells. The invention also evidences corneal epithelial differentiation by the expression of CK-19 and CK-3 after mix-culture with corneal epithelial cells while human adipose-derived stem cells from subcutaneous fat are unable to differentiate into corneal epithelial cells under the same induction condition.

Accordingly, in another aspect, the invention provides a method of regeneration of lost corneal epithelial cells on the ocular surface, comprising containing OFSCs with corneal epithelial cells. That is, the invention provides a use of OFSCs in the manufacture of a medicament for regenerating lost corneal epithelial cells on the ocular surface, wherein the OFSCs contact with corneal epithelial cells. In this connection, paracrine effects may not play a major role for corneal epithelial cells to induce epithelial commitment and corneal epithelial differentiation, as transwell culture of corneal epithelial cells was not able to exert the same induction effects as in the contact mix-culture. In a further aspect, the invention provides a kit for regeneration of lost corneal epithelial cells on the ocular surface, comprising the OFSCs and the corneal epithelial cells in separate packs. By using the method or the kit, cell therapy of corneal diseases due to the loss of corneal epithelial cells and tissue engineering of corneal epithelium may be achieved.

According to the invention, one or more cellular differentiation agents, such as cytokines and growth factors can be further used in the method and kit for regeneration of lost corneal epithelial cells.

### Method for Preparation of Corneal Epithelial Cell Preparations

In another further aspect, the invention provides a method for preparing corneal epithelial cell preparations, comprising: (a) isolating orbital fat-derived stem cells (OFSCs) from orbital adipose samples; (b) mix-culturing the OFSCs with labeled corneal epithelial cells to differentiating into corneal epithelial cells; and (c) removing the labeled corneal epithelial cells to obtain the OFSCs-derived corneal epithelial cell preparations.

According to the invention, the isolation of step (a) and mix-culture of step (b) are as mentioned herein.

According to the invention, effective separation of corneal epithelial cells from OFSCs-derived corneal epithelial progenies is mandatory. One solution is to add labeling to corneal epithelial cells, so as to increase the separation efficiency. Particularly, the corneal epithelial cells in step (b) are labeled. Any detectable label known in the art can be used. For example, a radio-isotope label, an enzyme label, a magnetic bead or a fluorescent label can be used.

According to the invention, in step (c), any method known in the art can be used to remove the labeled corneal epithelial cells from the OFSCs-derived corneal epithelial cells.

In one embodiment, before step (b), a step of expanding the OFSCs may be performed.

The ability of epithelial lineage commitment and differentiation into corneal epithelial cells indicates the potential clinical application of OFSCs in cell therapy of corneal diseases due to the loss of corneal/limbal epithelial cells.

The following examples are provided to demonstrate particular situations and settings in which this technology may be applied and are not intended to restrict the scope of the invention and the claims included in this disclosure.

### EXAMPLE

The following experimental examples are provided in order to demonstrate and further illustrate various aspects of certain embodiments of the present invention and are not to be construed as limiting the scope thereof. In the experimental disclosure which follows, the following materials and methods are used:

### 1. Antibodies

For flow cytometry, antibodies against human antigens CD10, CD29, CD31, CD34, CD44, CD49b, CD49d, CD49e, CD54, CD58, CD90, CD106, CD117, CD146, CD166, and HLA-DR were purchased from BD Biosciences (San Jose, Calif., USA). Antibodies against human antigen CD133 were purchased from Miltenyi Biotec (Bergisch Gladbach, Germany). Antibodies against human antigens CD14, CD45, and HLA-ABC were purchased from eBioscience (San Diego, Calif., USA). Antibodies against human antigen CD105 and epithelial specific antigen (ESA) were purchased from R& D system (Minneapolis, Minn., USA).

For immunofluorescence staining, rabbit antibody against human zonal occludin-1 (ZO-1) was purchased from Abcam (Cambridge, Mass., USA). Mouse anti-human cytokeratin 19 (CK19) and cytokeratin 3 (CK3) antibodies were purchased from Millipore (Billerica, Mass., USA). For Secondary antibodies, Cy3-conjugated sheep anti-rabbit IgG antibody was purchased from Sigma-Aldrich (St. Louis, Mo., USA), and Cy2-conjugated Goat against mouse IgG antibody was purchased from Jackson ImmunoResearch (West Grove, Pa., USA).

### 2. Isolation and Culture of Orbital Fat-Derived Stem Cells (OFSCs)

Under local anesthesia, redundant orbital fat tissues of healthy donors were removed from intraorbital cavity during blepharoplastic surgeries (n=5). One milliliter of orbital fat tissues was collected from each donor, and tissues were fragmented with surgical scissors and suspended in 0.1% collagenase type I (Worthington Biochemical Corporation, Lakewood, N.J., USA) in phosphate-buffered saline (PBS; Gibco, Grand Island, N.Y., USA) at 37° C. After 4-hour digestion, fragmented tissues were filtered through 70 μm strainer. The fluid was washed with PBS and centrifuged twice for 5 minutes at 1000 rpm at room temperature. After re-suspension of the pellet, cells were counted and plated in noncoated tissue culture flasks with seeding density of 3000-5000/cm². Cells were maintained in Mesen Pro Medium (Invitrogen, Carlsbad, Calif., USA) and allowed to adhere overnight and nonadherent cells were washed out with medium changes. The initial density of colony-forming cells was around 0.05/cm².

### 3. Isolation of Bone Marrow-Derived Mesenchymal Stem Cells (BM-MSCs) and Adipose-Derived Stem Cells (ADSCs)

BM-MSCs were isolated according to our previously reported protocol (Lee K D, Kuo T K, Whang-Peng J, et al. Hepatology. 2004; 40:1275-1284). Briefly, negative immuno-selection and limiting dilution were performed to isolate single cell-derived, clonally-expanded MSCs from the mononuclear fraction of bone marrow aspirates. ADSCs were isolated from the stromal vascular fraction of adipose tissues obtained during abdominal surgeries according to the protocols reported in the literature (Zuk P A, Zhu M, Mizuno H, et al. Tissue Eng. 2001; 7:211-228).

### 4. Maintenance and Expansion of Stem Cells

Once adherent cells reached approximately 60% to 70% confluence, they were detached with 0.25% trypsin-EDTA (ethylenediaminetetraacetic acid; Gibco), washed twice with PBS, centrifuged at 1000 rpm for 5 minutes, and re-plated at 1:3 under the same culture conditions. Cell numbers were counted as well as cumulative population doublings (PDs) and cumulative time were calculated in each passage. All the following experiments were performed by at least three independent donors (n>=3). BM-MSCs and ADSCs were also maintained and expanded in Mesen Pro Medium (Invitrogen) using the above mentioned protocol.

### 2. Surface Immuno-Phenotyping

For cell surface antigen immuno-phenotyping, sixth- to eighth-passage orbital fat-derived cells or human corneal epithelial cells (HCE-T cells) (Ho J H, Chuang C H, Ho C Y, Shih Y R, Lee O K, Su Y., Invest Ophthalmol Vis Sci. 2007; 48:27-33; Ho J H, Tseng K C, Ma W H, Chen K H, Lee O K, Su Y., Br J Ophthalmol. 2008; 92:992-7) were detached and stained with FITC- or PE-conjugated antibodies and analyzed with FACSCalibur (BD Biosciences).

### 6. In Vitro Differentiation and Evaluation of OFSCs

To induce in vitro differentiation, eighth- to tenth-passage orbital fat-derived cells were treated with osteogenic, chondrogenic, or adipogenic medium as previously described for bone marrow and umbilical cord blood-derived mesenchymal stem cells (Lee O K, Kuo T K, Chen W M, Lee K D, Hsieh S L, Chen T H., Blood. 2004; 103:1669-1675; Ho J H, Ma W H, Su Y, Tseng K C, Kuo T K, Lee O K., J Orthop Res. 2010; 28:131-138).

Histologic, cytochemical, and immunocytochemical analysis. For osteogenic differentiation, alkaline phosphatase staining was performed, and mineralized matrix was evaluated by von Kossa staining. For chondrogenic differentiation, pellets were fixed and embedded. The cutting sections were stained with hematoxylin and eosin (H&E) and Safranin O. For adipogenic differentiation, intracellular lipid droplets were stained with oil-red O. All staining protocols have been previously described elsewhere by the authors (Lee O K, Kuo T K, Chen W M, Lee K D, Hsieh S L, Chen T H., Blood. 2004; 103:1669-1675; Ho J H, Ma W H, Su Y, Tseng K C, Kuo T K, Lee O K., J Orthop Res. 2010; 28:131-138).

Total RNA isolation and real-time RT-PCR. RNA was extracted from 3×10⁵ OFSCs and differentiated cells for reverse transcription into cDNA and amplification as described previously (Ho J H, Ma W H, Su Y, Tseng K C, Kuo T K, Lee O K, J Orthop Res. 2010; 28:131-138). Primers used for real-time RT-PCR are listed in Table 1.

TABLE 1

| Gene | Primer Sequence | | Product |
|---|---|---|---|
| Osteogenic marker | F : agaaccccaaaggcttcttc | R: cttggcttttccttcatggt | 74 bp |
| genes | (SEQ ID NO: 1) | (SEQ ID NO: 2) | |
| Alkaline phosphatase | F: agaaccccaaaggcttcttc | R: acctttactggactctgcac | 98 bp |
| (ALP) | (SEQ ID NO: 3) | (SEQ ID NO: 4) | |
| osteocalcin | | | |
| Collagen, type I, | F: gggattccctggacctaaag | R: ggaacacctcgctctcca | 63 bp |
| alpha 1 (COL 1A1) | (SEQ ID NO: 5) | (SEQ ID NO: 6) | |
| Collagen, type I, | F: tctggagaggctggtactgc | R: gagcaccaagaagaccctga | 64 bp |
| alpha 2 (COL 1A2) | (SEQ ID NO: 7) | (SEQ ID NO: 8) | |
| Periostin, osteoblast | F: gaaccaaaaattaaagtgattgaagg | R: tgactttgttagtgtgggtcct | 76 bp |
| specific factor | (SEQ ID NO: 9) | (SEQ ID NO: 10) | |
| (POSTN) | | | |
| Osteopontin | F: gcttggttgtcagcagca | R: tgcaattctcatggtagtgagttt | 127 bp |
| | (SEQ ID NO: 11) | (SEQ ID NO: 12) | |
| Osteonectin | F: gtgcagaggaaaccgaagag | R: tgtttgcagtggtggttctg | 64 bp |
| | (SEQ ID NO: 13) | (SEQ ID NO: 14) | |
| Chondrogenic | | | |
| marker genes | | | |
| Aggrecan (ACAN) | F: tacactggcgagcactgtaac | R: cagtggccctggtacttgtt | 71 bp |
| | (SEQ ID NO: 15) | (SEQ ID NO: 16) | |
| Collagen, type II, | F: gtgtcagggccaggatgt | R: tcccagtgtcacagacacagat | 116 bp |
| alpha 1 (COL 2A1) | (SEQ ID NO: 17) | (SEQ ID NO: 18) | |
| Cartilage acidic | F: ggagtgtggccaagattc | R: gatgcattcattggtgtcca | 64 bp |
| protein 1 (CRTAC1) | (SEQ ID NO: 19) | (SEQ ID NO: 20) | |
| Cartilage oligomeric | F: gcaccgacgtcaacgagt | R: tggtgttgatacagcggact | 63 bp |
| matrix protein | (SEQ ID NO: 21) | (SEQ ID NO: 22) | |
| (COMP) | | | |
| Heparan sulfate | F: tctggctcaagtgctgtcc | R: gaggaggagggctcgatg | 71 bp |
| proteoglycan 2 | (SEQ ID NO: 23) | (SEQ ID NO: 24) | |
| (HSPG2) | | | |
| Matrillin 1, cartilage | F: atcgagaagctgtccaggaa | R: agtcatggtcccctggg | 76 bp |
| matrix protein | (SEQ ID NO: 25) | (SEQ ID NO: 26) | |
| (MATN1) | | | |
| Syndecan 2 (SDC2) | F: aaacggacagaagtcctagcag | R: aaattgcaaagagaaagccaa | 64 bp |
| | (SEQ ID NO: 27) | (SEQ ID NO: 28) | |
| Adipogenic marker | | | |
| genes | | | |
| Adipsin, complement | F: tccaagcgcctgtacgac | R: gtgtggccttctccgaca | 106 bp |
| factor D (CFD) | (SEQ ID NO: 29) | (SEQ ID NO: 30) | |
| Fatty acid synthase | F: caggcacacacgatggac | R: cggagtgaatctgggttgat | 92 bp |
| (FASN) | (SEQ ID NO: 31) | (SEQ ID NO: 32) | |
| Peroxisome | F: tccatgctgttatgggtgaa | R: tgtgtcaaccatggtcatttc | 113 bp |
| proliferator-activated | (SEQ ID NO: 33) | (SEQ ID NO: 34) | |
| receptor gamma | | | |
| (PPARγ) | | | |
| Adipocyte fatty acid | F: cctttaaaaatactgagatttccttca | R: ggacacccccatctaaggtt | 105 bp |
| binding protein (aP2) | (SEQ ID NO: 35) | (SEQ ID NO: 36) | |
| Leptin (LEP) | F: ttgtcaccaggatcaatgaca | R: gtccaaaccggtgactttct | 71 bp |
| | (SEQ ID NO: 37) | (SEQ ID NO: 38) | |
| Adiponectin | F: ggtgagaagggtgagaaagga | R: tttcaccgatgtctcccttag | 61 bp |
| | (SEQ ID NO: 39) | (SEQ ID NO: 40) | |
| Housekeeping gene | | | |
| GAPDH | F: agccacatcgctcagacac | R: gcccaatacgaccaaatcc | 66 bp |
| | (SEQ ID NO: 41) | (SEQ ID NO: 42) | |

### 7. Mix-Culture

OFSCs or ADSCs were seeded on 6-well plates with 2.9×10⁴ cells (30% confluence) and maintained in Mesen Pro medium overnight. On the next day, Mesen Pro medium was removed and 3.5×10⁴HCE-T cells (30% of confluence) were added into the plate. For the following five to seven days, cells were cultured in medium for HCE-T cells (Ho J H, Chuang C H, Ho C Y, Shih Y R, Lee O K, Su Y., Invest Ophthalmol Vis Sci. 2007; 48:27-33; Ho J H, Tseng K C, Ma W H, Chen K H, Lee O K, Su Y., Br J Ophthalmol. 2008; 92:992-7; Araki-Sasaki K, Ohashi Y, Sasabe T, et al., Invest Ophthalmol Vis Sci. 1995; 36:614-621) containing DMEM/HamF12 (1:1) medium supplemented with 5% fetal bovine serum (HyClone, Logan, Utah, USA), 5 μg/ml insulin, 0.1 μg/ml cholera toxin (Sigma-Aldrich), 10 ng/ml recombinant human epidermal growth factor (hEGF) (BD Biosciences), and 0.5% DMSO (Araki-Sasaki K, Ohashi Y, Sasabe T, et al., Invest Ophthalmol Vis Sci. 1995; 36:614-621).

### 8. OFSCs in Human Corneal Epithelial Cell Culture Medium

OFSCs were seeded on 6-well plate with 2.9×10⁴ cells (30% confluence) and maintained in Mesen Pro medium overnight. On the next day, Mesen Pro medium was removed and OFSCs were cultured in medium for HCE-T cells as the above described.

### 9. Transwell Culture

OFSCs were seeded on 6-well TC Plates (BD Falcon™ Cat. No. 353502) with 2.9×10⁴ cells (30% confluence) and HCE-T cells were seeded on 0.4 μm pore membrane of cell culture insert (BD Falcon™ Cat. No. 353090). Cells were cultured in medium for HCE-T cells as described above.

### 10. Quantum Dot Labeling

OFSCs were seeded on 6-well plate with 2.9×10⁴ cells (30% confluence) and maintained in Mesen Pro medium overnight. On the next day, OFSCs were incubated with quantum dots (Invitrogen) at various concentrations (1, 2, 5 and 10 nM) for 1 hour. After twice PBS washes, 3.5×10⁴ HCE-T cells (30% of confluence) were added into the plate with quantum dots labeled OFSCs.

### 11. Epithelial Phenotype Characterization

For ESA and CD105 detection, cells were detached, stained with FITC-conjugated antibodies and analyzed with FACSCalibur (BD Biosciences). For ZO-1 staining, cells were fixed in 4% formaldehyde for 20 minutes, followed by PBS wash twice. After blocked in 5% milk for 1 hour, cells were incubated with anti-ZO-1 (1:100) at room temperature for 1 hour, followed by a Cy3-conjugated anti-rabbit antibody (1:200) for another 30 minutes. At the end, nuclei were stained with 4,6-diamidino-2-phenylindole (DAPI), and cell images were assessed under a fluorescence microscope (Leitz, Germany). Imaging was performed with SPOT RT Imaging system (Diagnostic Instruments, Sterling Heights, Mich., USA).

### 12. Corneal Epithelial Phenotype Characterization

For CK19 and CK3 staining, cells were fixed in 4% formaldehyde for 20 minutes, followed by PBS wash twice. After blocked in 5% milk for 1 hour, cells were incubated with anti-CK19 (1:200) or anti-CK3 (1:200) at room temperature for 1 hour, followed by incubation with a Cy2-conjugated anti-mouse antibody (1:200) for 30 minutes. Nucleus was then stained with 4,6-diamidino-2-phenylindole (DAPI), and the samples were assessed under a fluorescence microscope (Leitz). Image acquisition was performed with SPOT RT Imaging system (Diagnostic Instruments).

### 13. Statistical Analysis

Statistical analysis was performed using the Statistical Package for Social Science-10 software (SPSS Inc., Chicago, Ill., USA). Changes of CD105 and ESA expressing cells in a mix-culture system were analyzed by ANOVA tests with Tukey's Post-Hoc tests at 95% confidence intervals. Different letters represent different levels of significance in the alphabetical order. Results of osteogenic, chondrogenic and adipogenic marker gene expressions as well as ESA expression in quantum dot-labeled cells were analyzed by two-tail, non-paired t tests, and P-values <0.05 were considered statistically significant.

### Example 1

### Characterization of Orbital Fat-Derived Stem Cells (OFSCs)

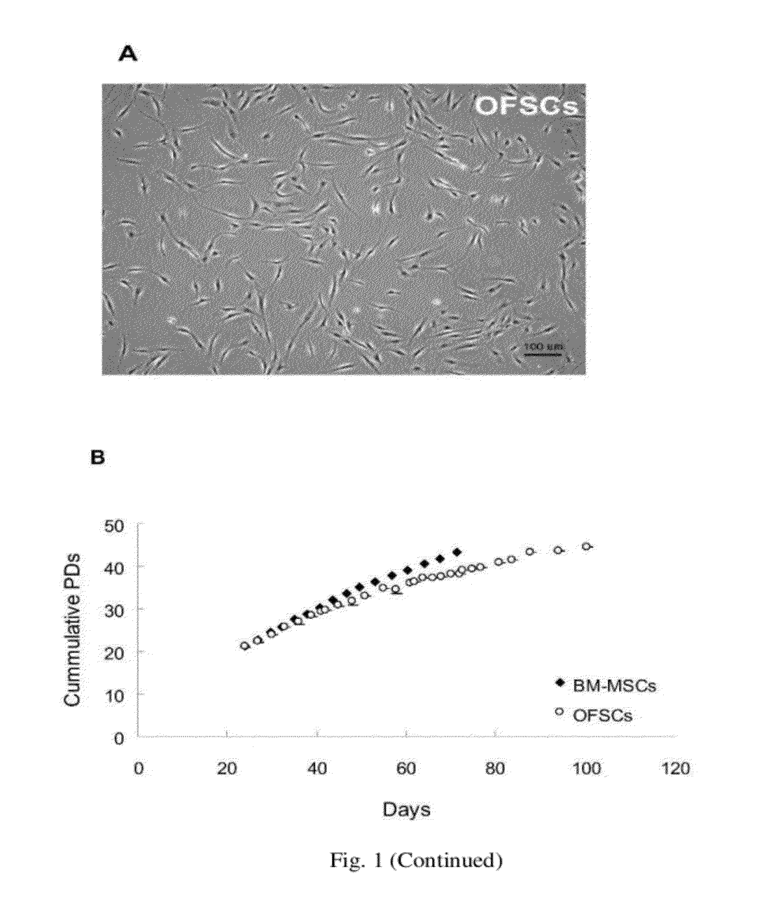

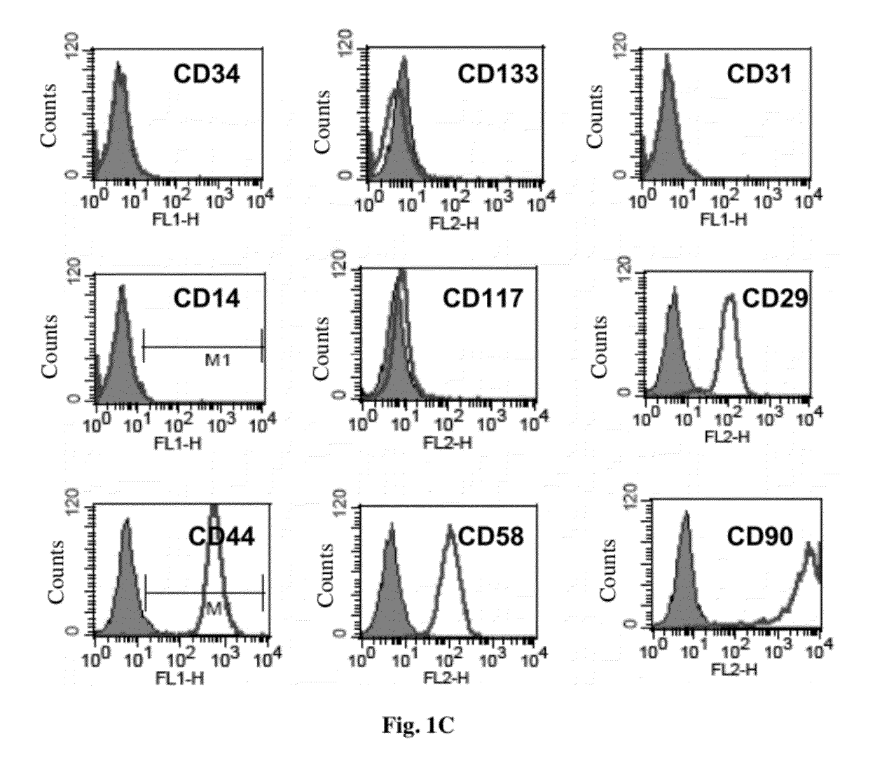

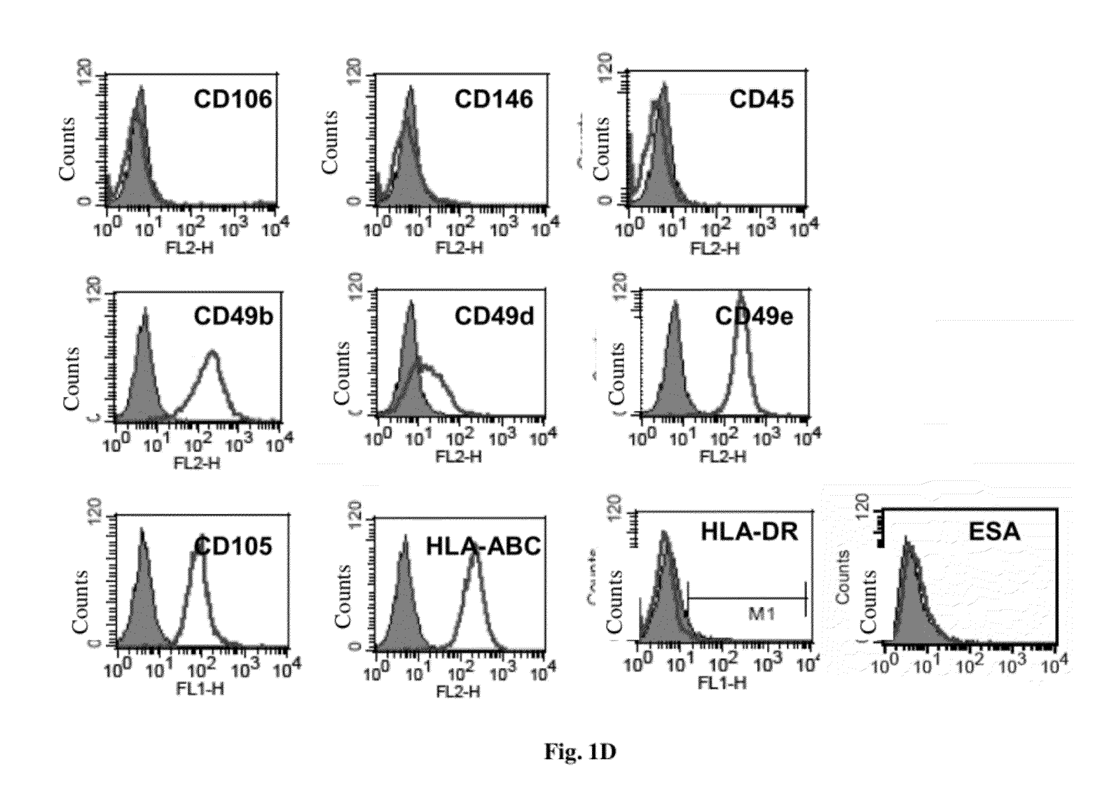

OFSCs were isolated from five donors (Male:Female=2:3) with the average age of 73.6 years. The frequency of colony-forming cells was 1/60,000-1/100,000. OFSCs were plastic-adherent, spindle-shaped, fibroblast-like cells (FIG. 1A). These cells could be extensively expanded for more than 45 cumulative population doublings, and growth kinetics curve of OFSCs was comparable to bone marrow-derived mesenchymal stem cells (BM-MSCs) (FIG. 1B). Surface immuno-phenotype characterized by flow cytometry revealed that OFSCs were negative for hematopoietic stem cell markers CD34, and CD133, endothelial progenitor cell marker CD31, vascular cell adhesion molecule-1 CD106, vascular endothelial tight junction marker CD146, leukocyte common antigen CD45, monocyte marker CD14 and CD117 (c-kit), indicating these cells were not of hematopoietic origin. OFSCs highly expressed β1 integrin CD29, α2 integrin CD49b, α5 integrin CD49e, matrix receptor CD44 and moderate expressed α4 integrin CD49d, suggesting their mesenchymal origin. Besides, these cells were positive for CD58 (LFA-3), CD90 (Thy-1), CD105 (endoglin), and expressed HLA-ABC but not HLA-DR (FIG. 1C), similar to the phenotype of BM-MSCs (Lee K D, Kuo T K, Whang-Peng J, et al., Hepatology. 2004; 40:1275-1284). Besides, lack of ESA expression (FIG. 1C) excluded the epithelial phenotype of these cells.

Expression of CD105 and CD 90 as well as the lack of hematopoietic and epithelial cell surface markers suggested that these cells were mesenchymal in nature (FIG. 1).

### Example 2

### Mesodermal Tri-Linage Differentiation of OFSCs

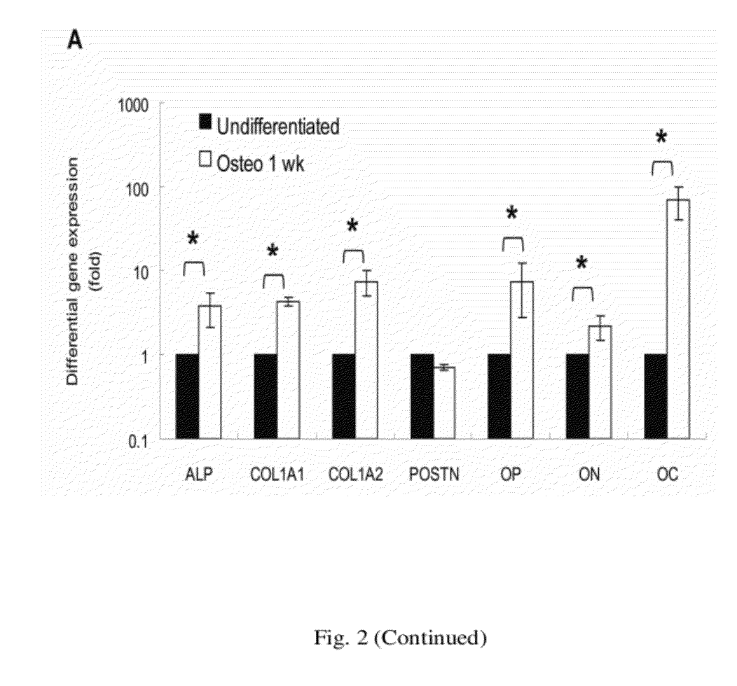

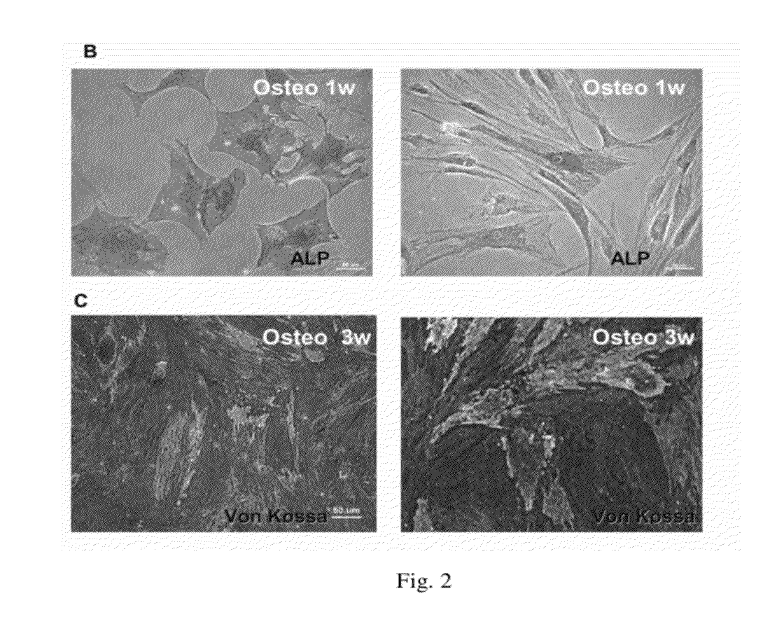

To test the tri-linage differentiation ability, the culture condition of OFSCs was shifted from Mesen Pro medium to induction medium. After one week of osteogenic induction, cells highly expressed osteogenic marker genes such as alkaline phosphatase (ALP), type I collagen α1 and α2 (Col IA1 and Col IA2), osteopontin (OP), osteonectin (ON) and osteocalcin (OC) (FIG. 2A) demonstrating the osteogenic commitment. Cells became more flattened and broadened in osteogenic medium (FIG. 2B) than that in Mesen Pro medium (FIG. 1A). Cells were positive for ALP staining after one-week induction (FIG. 2B), and were positive for von Kossa stain after three weeks of induction (FIG. 2C), showing their differentiation ability into mature osteoblasts.

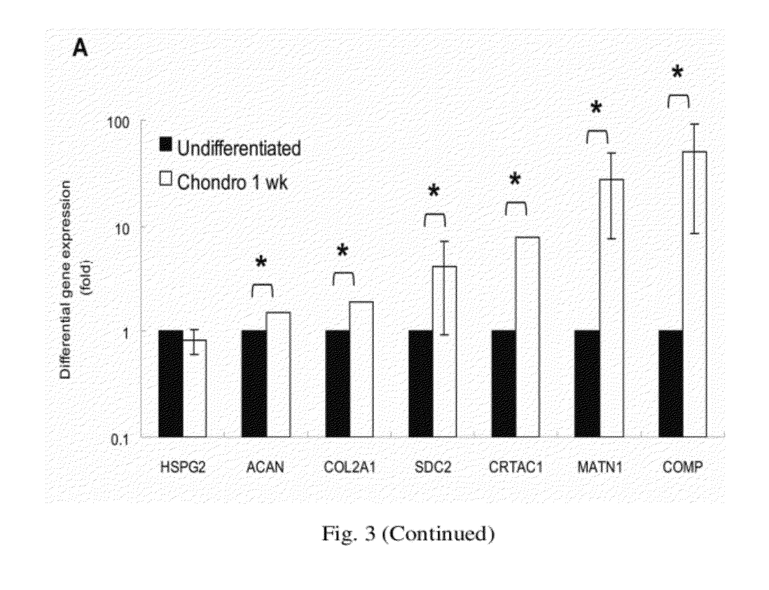

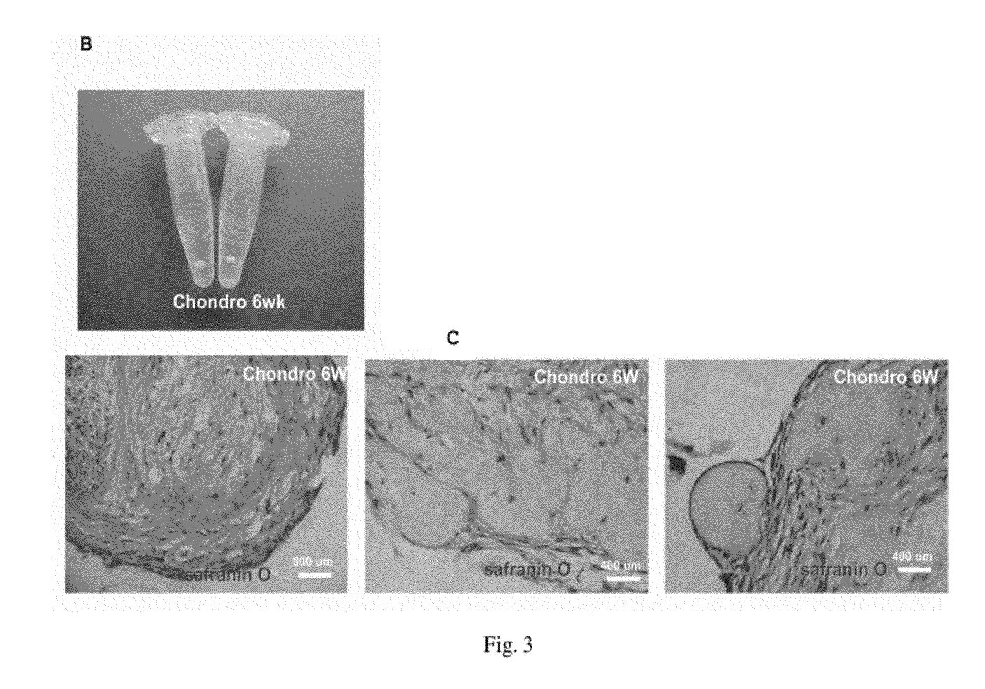

Chondrogenic differentiation ability was examined under pellet culture (FIG. 3B). After one-week chondrogenic induction, up-regulation of chondrogenic marker genes such as aggrecan (ACAN), Type II α1 collagen (Col IIA1), cartilage acidic protein 1 (CRTAC1), syndecan 2 (SDC2), cartilage oligomeric matrix protein (COMP) and cartilage matrix protein matrilin (MATN1) (FIG. 3A) indicated the chondrogenic potential. Expression of heparan sulfate proteoglycan 2 (HSPG2), a late chondrogenic marker, remained unchanged. Six weeks later, the section of pellets showed the accumulation of extracellular matrix stained by safranin O (FIG. 3C), indicating their differentiation ability into mature chondrocytes.

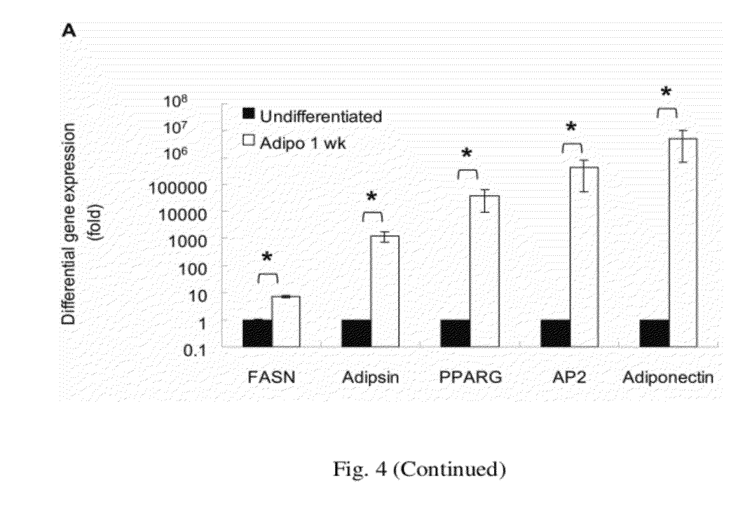

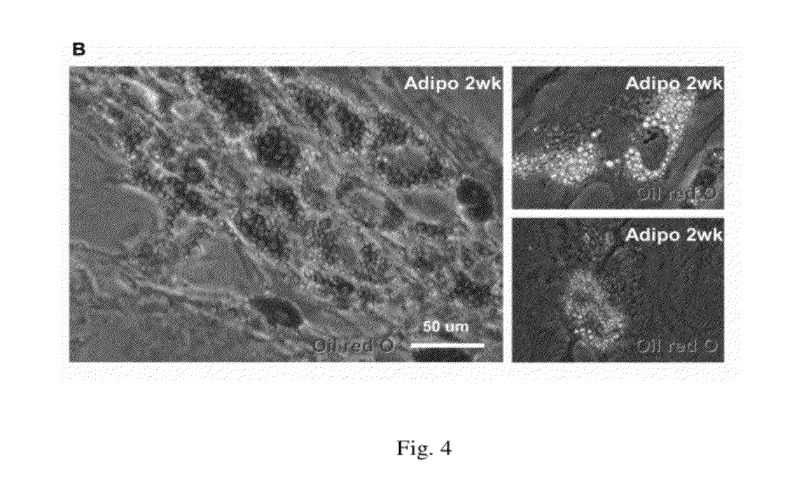

Under adipogenic induction, upregulation of peroxisome proliferator-activated receptor gamma (PPARgamma) indicating the adipogenic fate commitment (FIG. 4A). At the end of one-week induction, not only adipogenic marker genes such as fatty acid binding protein (aP2), fatty acid synthase (FASN) and complement factor D (Adipsin), but also adiponectin, were expressed in differentiated cells. Notably, the expression of these adipogenic marker genes was extremely high upon adipogenic induction (FIG. 4A). Besides, massive intracellular lipid droplets could be easily visible by oil red O staining after two weeks of induction (FIG. 4B), suggesting their differentiation ability into mature adipocytes. The baseline expression level of adipohormones in OFSCs such as adiponectin and leptin were very low (data not shown), while OFSCs-differentiated cells expressed extremely high level of adiponectin (FIG. 4A) rather than leptin (data not shown) during early adipogenic differentiation. Adiponectin, which is down-regulated in obesity, is known to enhance insulin sensitivity by lowering glucose production in liver, increasing glucose uptake and fatty acid oxidation in skeletal muscles, and inhibiting inflammatory reactions.

OFSCs possess the tri-lineage differentiation ability as they could differentiate into osteoblasts (FIG. 2), chondrocytes (FIG. 3) and adipocytes (FIG. 4). Comparing the differentiation potentials of OFSCs and BM-MSCs, osteogenic differentiation ability was similar. It took three to four weeks of both OFSCs (FIG. 2) and BM-MSCs (Zuk P A, Zhu M, Ashjian P, et al., Mol Biol Cell. 2002; 13:4279-4295; Zuk P A, Zhu M, Mizuno H, et al., Tissue Eng. 2001; 7:211-228) to differentiate into mature osteoblasts which produced mineralized matrix. For chondrogenic potential, under pellet culture for six weeks, both BM-MSCs24 and OFSCs (FIG. 3) differentiated into mature chondrocytes with the production of abundant extracellular matrix. For adipogenesis, it took at least three weeks for BM-MSCs to differentiate into mature adipocytes with intracellular lipid droplets accumulation (Lee K D, Kuo T K, Whang-Peng J, et al., Hepatology. 2004; 40:1275-1284; Ho J H, Ma W H, Su Y, Tseng K C, Kuo T K, Lee O K., J Orthop Res. 2010; 28:131-138). However, for OFSCs, the greater adipogenic differentiation potential was demonstrated by extremely high (>104 folds) up-regulation of adipocyte marker genes during the first week of adipogenic induction (FIG. 4A), which was accompanied by rapid and massive accumulation of intracellular lipid droplets (FIG. 4B) within the first two-weeks of adipogenic induction.

### Example 3

### Epithelial Differentiation of OFSCs

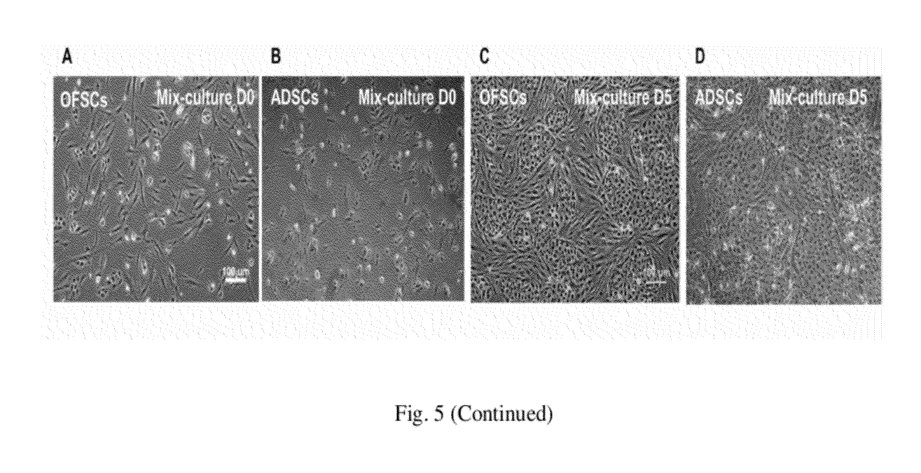

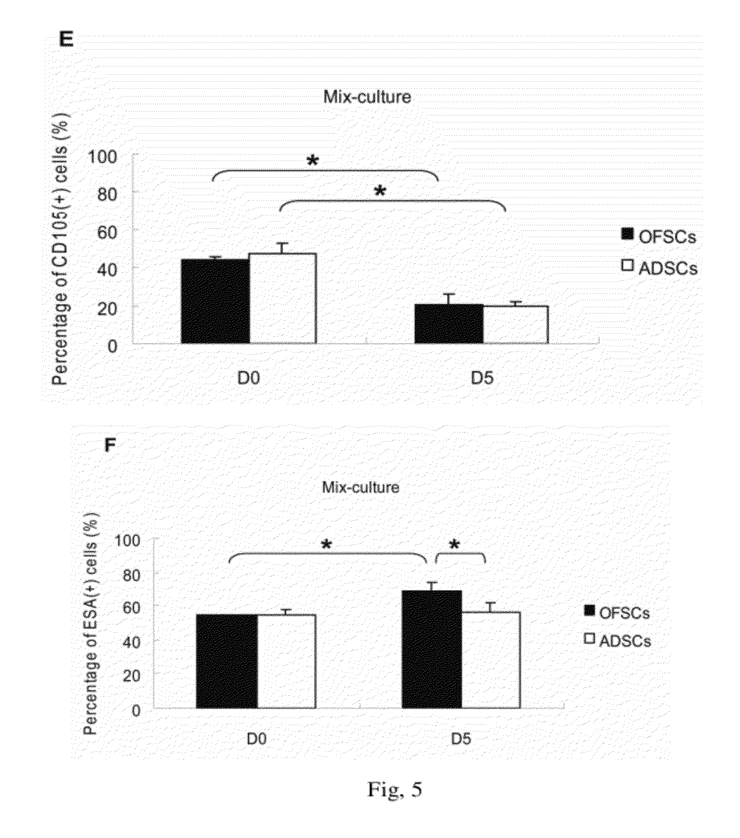

To investigate the difference of epithelial differentiation potential between OFSCs and ADSCs, OFSCs (FIG. 5A) as well as ADSCs (FIG. 5B) were mix-cultured with HCE-T cells in HCE-T medium. After 5-day of mix-culture, cells almost became confluent (FIGS. 5C and D). The frequency of CD105-positive cells was significantly reduced in both OFSCs and ADSCs after a 5-day mix-culture with HCE-T cells (FIG. 5E). However, the percentage of ESA-positive significantly increased in OFSCs only (FIG. 5F), suggesting significant mesenchymal to epithelial shifting of the phenotype only occurred in OFSCs but not in ADSCs.

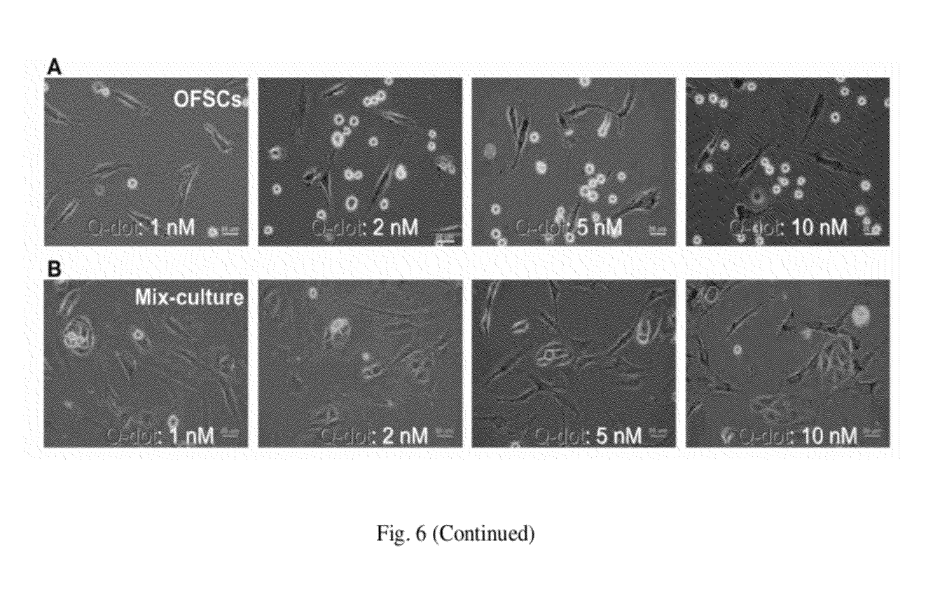

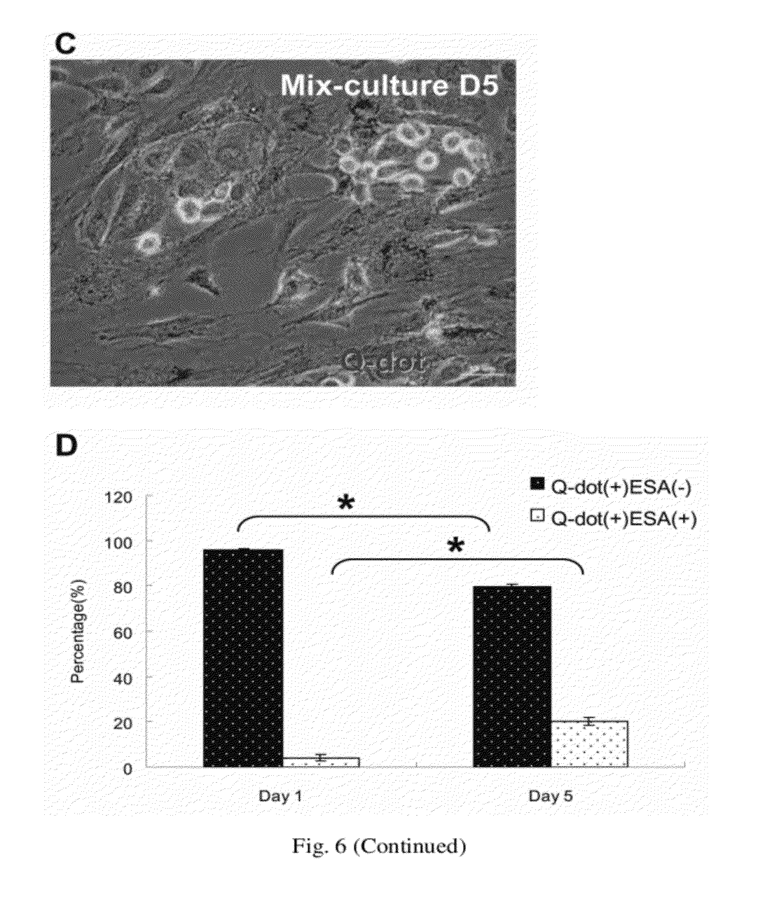

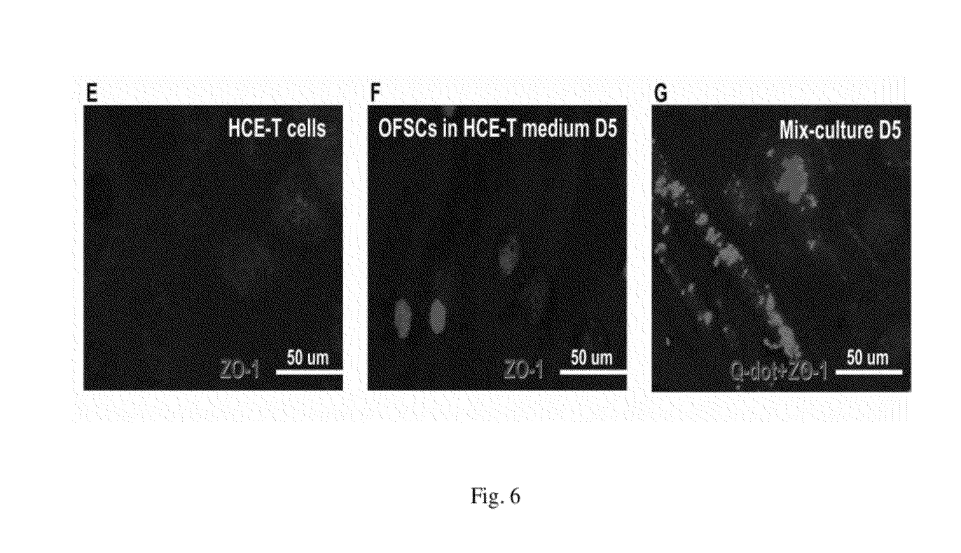

Next, to directly demonstrate the shift of phenotype into epithelial cells, OFSCs were labeled with quantum dots. First, dose-dependent labeling efficiency was shown in FIG. 6A; quantum dot-labeled OFSCs with red fluorescence signals can be easily distinguished from cobblestone-like HCE-T cells in the mix-culture (FIG. 6B). After 5 days of mix-culture, a proportion of quantum dot-labeled cells, particularly those surrounded cobblestone-like HCE-T cells, became oval to round in shape (FIG. 6C). Flow cytometric analysis showed that 20.1±0.77% of quantum dot-labeled cells began to express ESA after 5 days of mix-culture (FIG. 6D). Moreover, ZO-1, the marker of epithelial tight junction which was expressed in HCE-T cells (FIG. 6E) but not in OFSCs cultured in HCE-T medium (FIG. 6F), became detectable in the junctions between quantum dot-labeled cells and their neighboring cells after 5 days of mix-culture (FIG. 6G). The above finding demonstrated that OFSCs possess the potential to differentiate into epithelial cells.

### Example 4

### Differentiating Potentials of OFSCs into Corneal Epithelial Cells

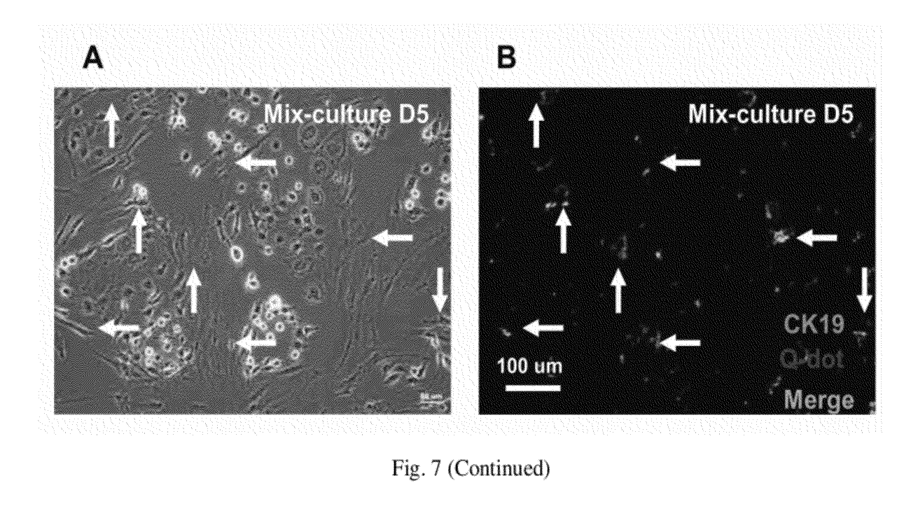

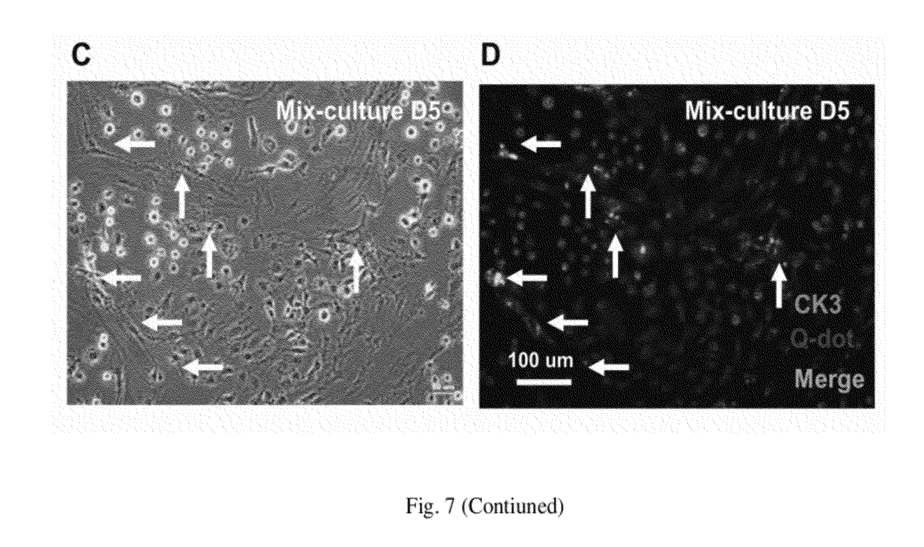

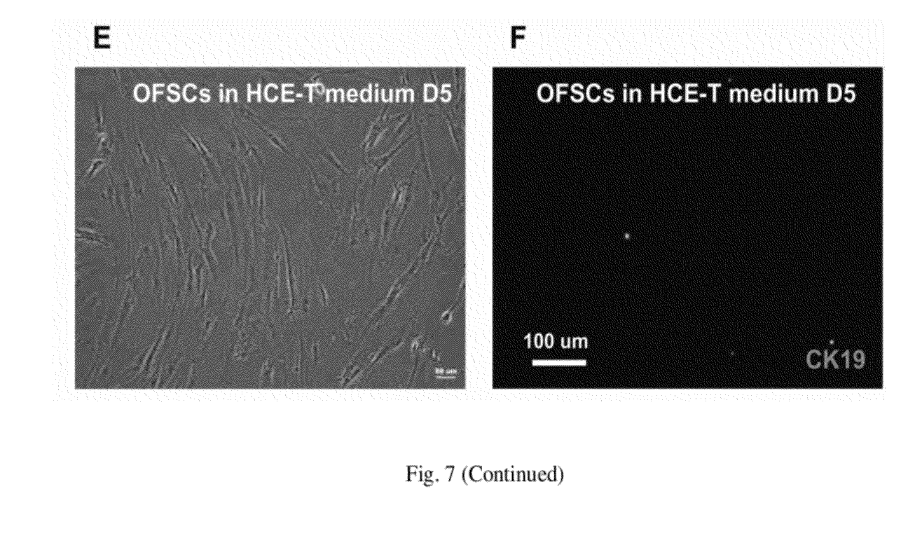

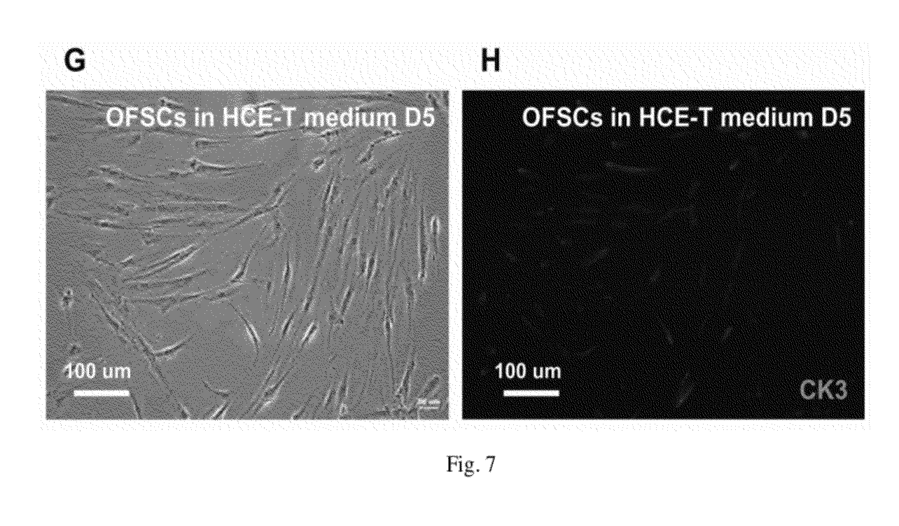

To further investigate whether OFSCs possessed the differentiation potentials into corneal epithelial cells, quantum dot-labeled OFSCs were mix-cultured with HCE-T cells for 5 days and the expression of CK19, the marker for corneal epithelial progenitors as well as CK3, the marker for mature corneal epithelial cells (Kinoshita S, Adachi W, Sotozono C, et al., Prog Retin Eye Res. 2001; 20:639-673) which was expressed in HCE-T cells (Araki-Sasaki K, Ohashi Y, Sasabe T, et al., Invest Ophthalmol Vis Sci. 1995; 36:614-621), was detected by immunofluorescence staining. It was found that, after mix-culture, some of quantum-dot labeled cells which are in contact with HCE-T cells expressed CK19, and no CK19 expression was found in any HCE-T cells (FIGS. 7A and B). CK3 expression was also detectable in some of quantum-dot labeled cells and was highly expressed in HCE-T cells (FIGS. 7C and D). To investigate whether co-culture with HCE-T cells is essential for OFSCs to express corneal epithelial phenotype, OFSCs alone were cultured under the same condition without HCE-T cells for 5 days. It was found that the morphology of OFSCs was not altered without HCE-T cells, and CK19 and CK3 was not induced either (FIGS. 7E to H).

In the mix-culture system with corneal epithelial cells, OFSCs rapidly expressed epithelial phenotype (FIGS. 5 and 6) as well as corneal epithelial phenotype (FIG. 7).

### Example 5

### Low Differentiation Potentials ADSCs into Corneal Epithelial Cells

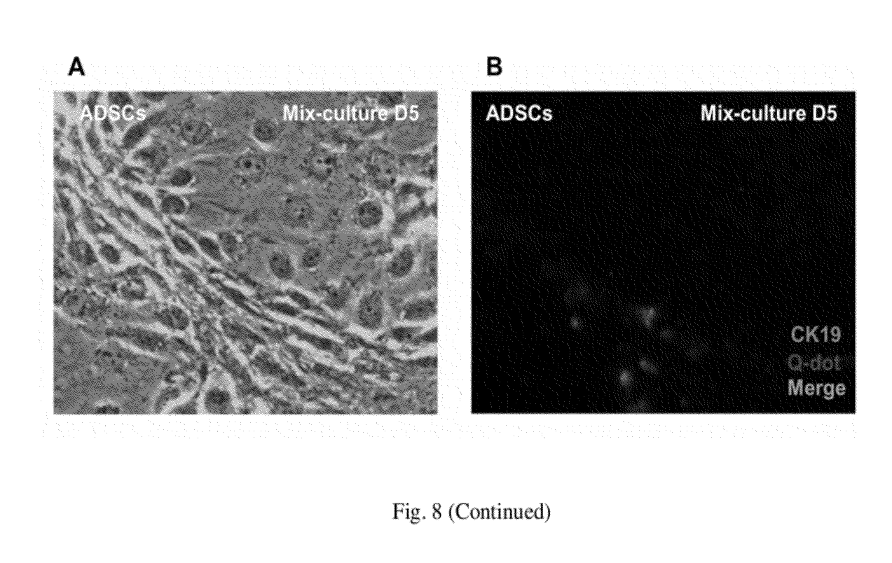

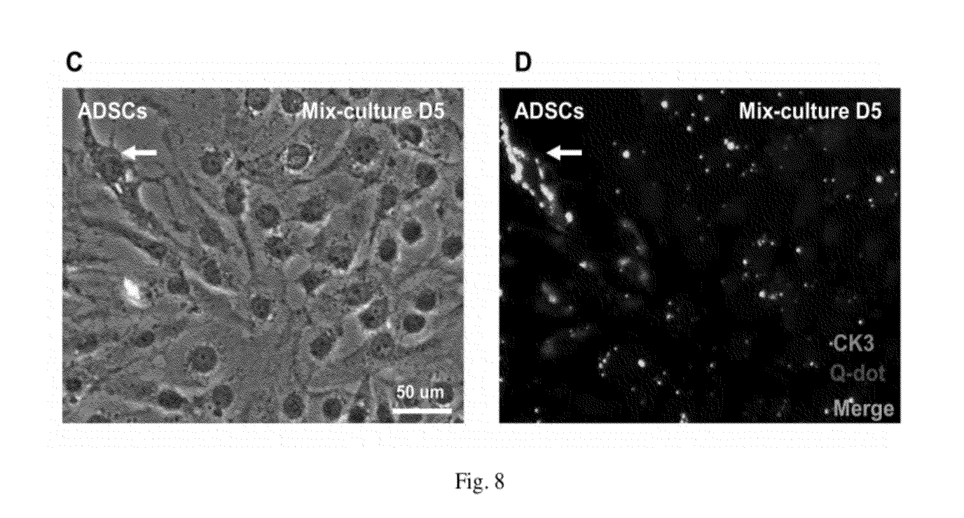

To investigate whether ADSCs could also differentiate into corneal epithelial cells, similar co-culture experiments of ADSCs and HCE-T cells were performed. However, quantum-dot labeled ADSCs did not express CK19 (FIGS. 8A and B), and only few quantum-dot labeled ADSCs at the margin of HCE-T cell islets expressed CK3 (FIGS. 8C and D) after mix-culture with HCE-T cells for 5 days. The percentage of CK3 expression is much lower in ADSCs in comparison with OFSCs (FIG. 7D).

Besides mesodermal tri-linage differentiation, epithelial differentiation potential of OFSCs has also been demonstrated in vitro through a mix-culture system (FIGS. 6 and 7). When OFSCs were co-cultured with HCE-T cells in a contact fashion, cells in mixed population shifted to epithelial phenotype evidenced by the dramatic loss of CD105 (FIG. 5E) and marked increase in ESA expression (FIG. 5F) during the first week of mix-culture. After mix-culture for 1 day, the decrease in ESA population (FIG. 6D) ruled out loss of OFSCs and overgrowth of HCE-T cells. It was intriguing that both morphological changes and the appearance of ZO-1 in quantum dot-labeled OFSCs (FIGS. 6C and 6G) in the mixed culture system were located at the contiguous area where OFSCs were in contact with HCE-T cell islands. However, neither significant morphological change nor ZO-1 expression could be observed in OFSCs (FIG. 6F) when they were cultured in HCE-T medium alone. The ability of corneal epithelial differentiation as evidenced by the expression of CK19 and CK3 (FIGS. 7A to 7D) indicates the therapeutic potentials of OFSCs to replenish lost corneal epithelial cells. Notably, ADSCs from subcutaneous fat tissues are very difficult to commit to epithelial lineage and differentiate into corneal epithelial cells upon mix-culture (FIGS. 5 and 8). Such findings have confirmed that OFSCs have the potential to differentiate into corneal epithelial cells due to the same developmental origin during embryonic development.

### Example 6

### Direct Contact with HCE-T Cells Indispensable for Epithelial Phenotype Induction of OFSCs

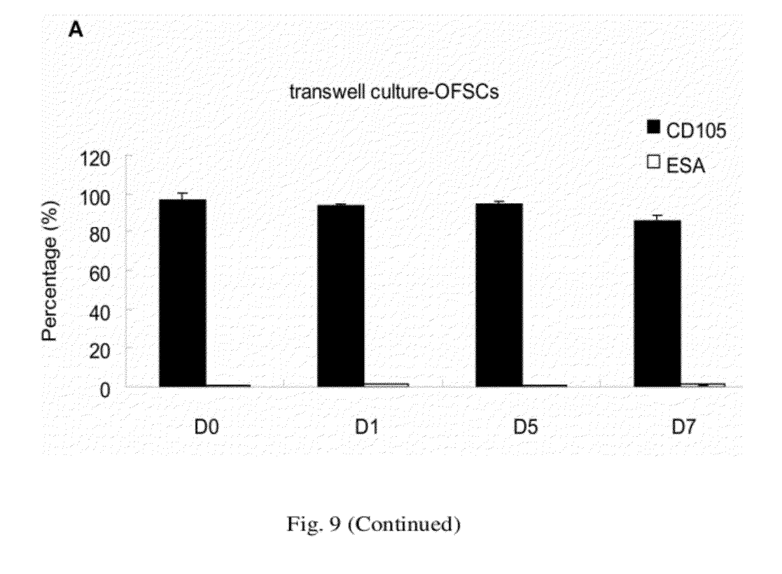

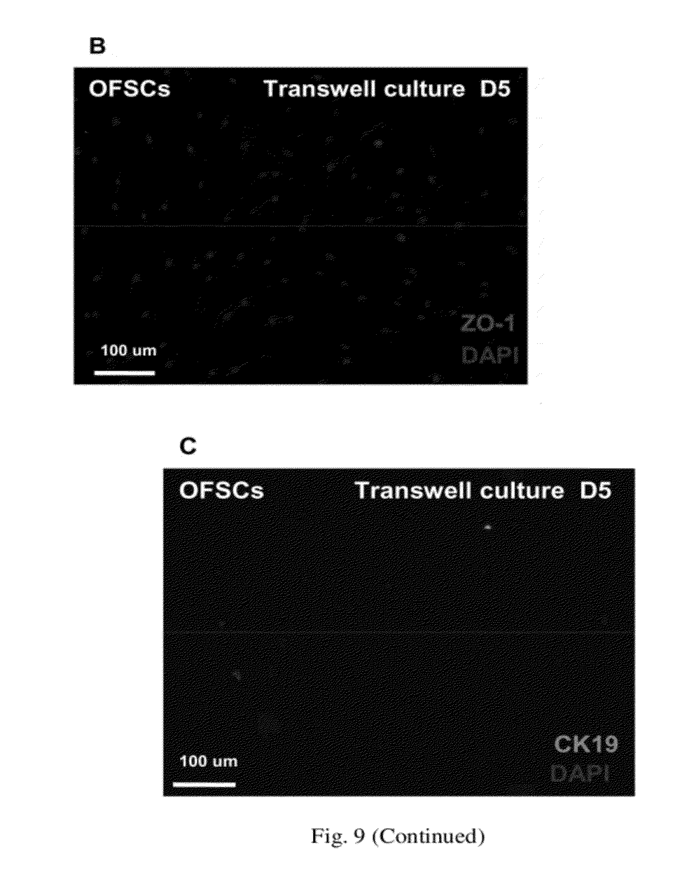

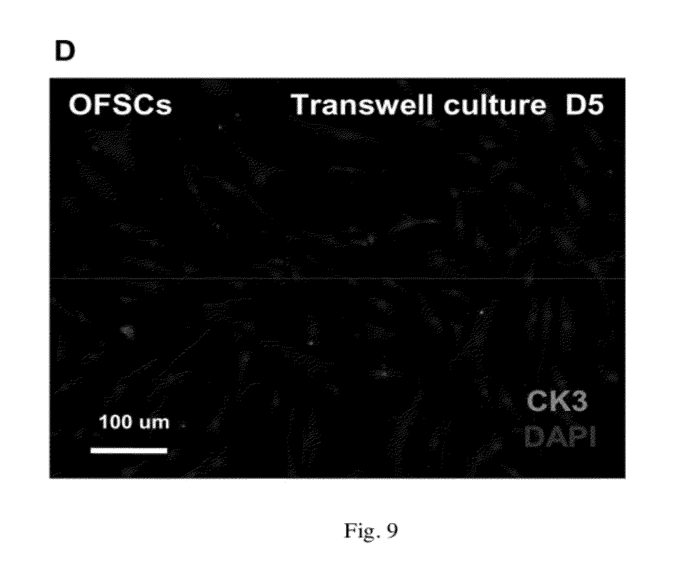

It was found that direct mix-culture with HCE-T cells induced epithelial differentiation of OFSCs. To investigate whether direct cell-contact between OFSCs and HCE-T cells was essential for such phenomenon, they were put in transwell non-contact co-culture for 7 days. It was found that expression of CD105 and ESA in OFSCs was not altered by transwell co-culture with HCE-T cells (FIG. 9A). The epithelial marker ZO-1 (FIG. 9B), and corneal epithelial markers CK19 (FIG. 9C) and CK3 (FIG. 9D), were not induced by transwell co-culture either.

Besides, it also demonstrated the crucial role of direct cell contact between OFSCs and HCE-T cells for epithelial differentiation of OFSCs (FIGS. 7 and 9), suggesting their potential for regeneration of lost corneal epithelial cells on the ocular surface via contact with corneal epithelial cells.

What is claimed is:

1. A cell population, which comprises orbital fat-derived stem cell (OFSCs) expressing at least CD90 and CD 105; wherein said OFSCs are not of hematopoietic and epithelial origins, and wherein said OFSCs are capable of multilineage development.

2. The cell population of claim 1, wherein the OFSCs further express CD29, CD44, CD49b, CD49e, CD58, HLA-ABC or a combination thereof.

3. The cell population of claim 1, wherein the OFSCs express CD29, CD44, CD49b, CD49e, CD58, CD90, CD105 and HLA ABC.

4. The cell population of claim 1, wherein the OFSCs do not express at least hematopoietic stem cell marker CD34.

5. The cell population of claim 1, wherein the OFSCs do not express CD 34 and CD 133.

6. The cell population of claim 5, wherein the OFSCs further do not express CD133, CD31, CD106, CD146, CD45, CD14, CD117 or a combination thereof.

7. The cell population of claim 1, wherein the OFSCs do not express CD40, CD80, CD86, HLA-DR or a combination thereof.

8. The cell population of claim 1, wherein the OFSCs have osteogenic, chondrogenic, adipogenic and corneal differentiation potentials.

9. The cell population of claim 1, wherein the OFSCs are mesenchymal origins but not hematopoietic and epithelial origins.

10. The cell population of claim 1, which can be used in cell therapy and tissue engineering.

11. The cell population of claim 1, which can be used in tissue regeneration for degenerative disease, repair of tissue injury, organ regeneration and medical cosmetology.

12. A composition, comprising the cell population of claim 1.

13. A method for isolation and purification of cell population comprising OFSCs of claim 1, comprising the steps of:
(a) collecting a sample containing 0.5-2 ml of orbital fat tissues;
(b) fragmenting the orbital fat tissues and suspending the resulting tissues in a buffer solution containing an extracellular matrix (ECM)-degrading enzyme;
(c) filtering the resulting solution to obtain the pellet;
(d) re-suspending the pellet to obtain a cell suspension solution;
(e) counting the cells in the cell suspension solution and culturing the cells in medium with low seeding density of less than 8,000 cells/cm²;
(f) collecting cells with colony-formation ability and sub-culturing these cells in an non-contact manner; and
(g) identifying and charactering the resulting cells with cell surface markers and multiple differentiation ability; wherein OFSCs are the resulting cells having multilineage development and expressing at least CD90 and CD 105 but lacking hematopoietic and epithelial cell surface markers.

14. The method of claim 13, wherein the sample is step (a) can be collected by directly removing the orbital tissue from intraorbital cavity or collected during blepharoplasty surgeries for entropion, ectropion, ptosis or baggy lid.

15. The method of claim 13, wherein the sample is step (a) contains about 0.5 to about 1.5 ml or about 0.5 to about 1.0 ml of orbital fat tissues.

16. The method of claim 13, wherein the seeding density is step (e) is from 500 to 8,000 cells/cm².

17. The method of claim 13, wherein the seeding density is step (e) is from 1,000 to 8,000 cells/cm².

18. The method of claim 13, wherein the seeding density is step (e) is from 3,000 to 5,000 cells/cm².

19. A method for differentiation of orbital fat-derived stem cells (OFSCs) to corneal epithelial cells, comprising the step of mix-culturing OFSCs with corneal epithelial cells.

20. The method of claim 19, wherein the OFSCs lose CD105 expression and increase expression of epithelial cell markers upon mix-culture with corneal epithelial cells.

21. The method of claim 20, wherein the epithelial cell markers include epithelial specific antigen and zonal occludin-1.

22. The method of claim 19, wherein the corneal epithelial differentiation of OFSCs is indicated by the expression of CK-19 and CK-3.

23. A method of regeneration of lost corneal epithelial cells on the ocular surface, comprising containing OFSCs with corneal epithelial cells.

24. A method for preparing corneal epithelial cell preparations, comprising: (a) isolating orbital fat-derived stem cells (OFSCs) from orbital adipose samples; (b) mix-culturing the OFSCs with labeled corneal epithelial cells to differentiating into corneal epithelial cells; and (c) removing the labeled corneal epithelial cells to obtain the OFSCs-derived corneal epithelial cell preparations.

25. The method of claim 24, wherein the label used to label the corneal epithelial cells in step (b) is a radio-isotope label, an enzyme label, a magnetic bead or a fluorescent label.


SEQUENCE LISTING (38 sequences)

SEQ ID NO: 1 - DNA, 20 bp, Artificial Sequence
  primer
agaaccccaaaggcttcttc

SEQ ID NO: 2 - DNA, 20 bp, Artificial Sequence
  primer
cttggcttttccttcatggt

SEQ ID NO: 3 - DNA, 20 bp, Artificial Sequence
  primer
tgagagccctcacactcctc

SEQ ID NO: 4 - DNA, 20 bp, Artificial Sequence
  primer
acctttgctggactctgcac

SEQ ID NO: 5 - DNA, 20 bp, Artificial Sequence
  primer
gggattccctggacctaaag

SEQ ID NO: 6 - DNA, 18 bp, Artificial Sequence
  primer
ggaacacctcgctctcca

SEQ ID NO: 7 - DNA, 20 bp, Artificial Sequence
  primer
tctggagaggctggtactgc

SEQ ID NO: 8 - DNA, 20 bp, Artificial Sequence
  primer
gagcaccaagaagaccctga

SEQ ID NO: 9 - DNA, 26 bp, Artificial Sequence
  primer
gaaccaaaaattaaagtgattgaagg

SEQ ID NO: 10 - DNA, 23 bp, Artificial Sequence
  primer
tgacttttgttagtgtgggtcct

SEQ ID NO: 11 - DNA, 18 bp, Artificial Sequence
  primer
gcttggttgtcagcagca

SEQ ID NO: 12 - DNA, 24 bp, Artificial Sequence
  primer
tgcaattctcatggtagtgagttt

SEQ ID NO: 13 - DNA, 20 bp, Artificial Sequence
  primer
gtgcagaggaaaccgaagag

SEQ ID NO: 14 - DNA, 20 bp, Artificial Sequence
  primer
tgtttgcagtggtggttctg

SEQ ID NO: 15 - DNA, 21 bp, Artificial Sequence
  primer
tacactggcgagcactgtaac

SEQ ID NO: 16 - DNA, 20 bp, Artificial Sequence
  primer
cagtggccctggtacttgtt

SEQ ID NO: 17 - DNA, 18 bp, Artificial Sequence
  primer
gtgtcagggccaggatgt

SEQ ID NO: 18 - DNA, 22 bp, Artificial Sequence
  primer
tcccagtgtcacagacacagat

SEQ ID NO: 19 - DNA, 19 bp, Artificial Sequence
  primer
ggagtgtggccaaggattc

SEQ ID NO: 20 - DNA, 20 bp, Artificial Sequence
  primer
gatgcattcattggtgtcca

SEQ ID NO: 21 - DNA, 18 bp, Artificial Sequence
  primer
gcaccgacgtcaacgagt

SEQ ID NO: 22 - DNA, 20 bp, Artificial Sequence
  primer
tggtgttgatacagcggact

SEQ ID NO: 23 - DNA, 19 bp, Artificial Sequence
  primer
tctggctcaagtgctgtcc

SEQ ID NO: 24 - DNA, 18 bp, Artificial Sequence
  primer
gaggaggagggctcgatg

SEQ ID NO: 25 - DNA, 20 bp, Artificial Sequence
  primer
atcgagaagctgtccaggaa

SEQ ID NO: 26 - DNA, 18 bp, Artificial Sequence
  primer
agtcatggtcccctgtgg

SEQ ID NO: 27 - DNA, 22 bp, Artificial Sequence
  primer
aaacggacagaagtcctagcag

SEQ ID NO: 28 - DNA, 22 bp, Artificial Sequence
  primer
aaattgcaaagagaaagccaat

SEQ ID NO: 29 - DNA, 18 bp, Artificial Sequence
  primer
tccaagcgcctgtacgac

SEQ ID NO: 30 - DNA, 18 bp, Artificial Sequence
  primer
gtgtggccttctccgaca

SEQ ID NO: 31 - DNA, 18 bp, Artificial Sequence
  primer
caggcacacacgatggac

SEQ ID NO: 32 - DNA, 20 bp, Artificial Sequence
  primer
cggagtgaatctgggttgat

SEQ ID NO: 33 - DNA, 20 bp, Artificial Sequence
  primer
tccatgctgttatgggtgaa

SEQ ID NO: 34 - DNA, 21 bp, Artificial Sequence
  primer
tgtgtcaaccatggtcatttc

SEQ ID NO: 35 - DNA, 27 bp, Artificial Sequence
  primer
cctttaaaaatactgagatttccttca

SEQ ID NO: 36 - DNA, 20 bp, Artificial Sequence
  primer
ggacacccccatctaaggtt

SEQ ID NO: 37 - DNA, 21 bp, Artificial Sequence
  primer
ttgtcaccaggatcaatgaca

SEQ ID NO: 38 - DNA, 20 bp, Artificial Sequence
  primer
gtccaaaccggtgactttct